(19) United States
(12) Patent Application Publication
(10) Pub. No.: US 2024/0013667 A1
(43) Pub. Date: Jan. 11, 2024

(54) System and Method for Facilitating Accessible Therapeutic Intervention to Regulate Emotions and Establish Immediate and Long-Term Well-Being by Synergizing Biophilic Stimuli with Cognitive Engagement to Amplify Benefits

(71) Applicant: Stacy L. Coleman, New York, NY (US)

(72) Inventor: Stacy L. Coleman, New York, NY (US)

(21) Appl. No.: 18/350,483

(22) Filed: Jul. 11, 2023

Related U.S. Application Data
(60) Provisional application No. 63/368,082, filed on Jul. 11, 2022.

(51) Int. Cl. G09B 5/04; G06F 3/16; G06K 7/14; G06K 19/06; A61M 21/00

(52) U.S. Cl. CPC G09B 5/04; G06F 3/165; G06K 7/1417; G06K 19/06037; A61M 21/00; A61M 2021/0027

(57) ABSTRACT

A system and a method for facilitating accessible therapeutic intervention to regulate emotions and establish immediate and long-term well-being by synergizing biophilic stimuli with cognitive engagement. The system includes a biophilic sensory unit with a matrix barcode associated with a corresponding uniform resource locator (URL) link. The system also includes audio recordings managed by a remote server. The method includes the steps of prompting a user account to scan the matrix barcode. The corresponding URL link is output, if the matrix barcode is scanned. The user account is then prompted to open the corresponding URL link. An audio recording is relayed from the remote server, when the corresponding URL link is opened. Then, the audio recording is output to allow the user to shift from their current state of mind and enter a new state of positive emotions by interacting with the biophilic sensory unit according to the audio recording.

### FIELD OF THE INVENTION

The present invention relates to systems and methods for biophilic therapy. More specifically, the present invention provides means to integrate established, effective tools of psychology and biophilic experiences to provide the user with both significant immediate stress reduction, calm, and well-being as well as lifelong skills for ongoing emotional health and well-being.

### BACKGROUND OF THE INVENTION

Work environments, especially in office settings, can be burdensome and stressful to many people. Unhappy, stressed, and overworked employees often lead to low engagement, low productivity, and poor overall performance. Accordingly, mental health is now seen as important as physical health. As a result, several tools have been made available in many workplaces for employees to relieve some stress and to wind down in the workplace. For example, gyms, game rooms, and other options of leisure and stress relief are conventional means of addressing this issue in the workplace. However, these conventional solutions can often be expensive, ineffective, and can take up a lot of office space. More importantly, the current tools provide only temporary relief, and do not address underlying issues or provide lasting solutions. Therefore, there is a need for new and innovative means of addressing unhappiness at its core, providing access to psychological health tools, as well as providing meaningful stress relief in the workplace.

An objective of the present invention is to provide means for facilitating accessible therapeutic intervention to regulate emotions, establish well-being, and provide lifelong behavioral and cognitive skills by synergizing biophilic stimuli with cognitive engagement. The present invention uses the established positive psychological effects of the biophilic experience to amplify cognitive engagement for optimally incorporating learned behavioral and cognitive skills. This results in releasing unwanted emotions, restoring a state of calm and well-being for the user, and providing a skillset to prepare the user to cope with future challenges. The present invention incorporates sight, sound and spoken word cues to achieve the aforementioned results. Additional features and benefits of the present invention are further discussed in the sections below.

### SUMMARY OF THE INVENTION

The present invention provides a system and a method for facilitating accessible therapeutic intervention to regulate emotions, establish well-being, and provide lifelong behavioral and cognitive skills by synergizing biophilic stimuli with cognitive engagement. The present invention combines the lush visuals of preserved moss, ferns, or living plants (i.e., biophilic stimuli) with connections to professionally scripted journeys of emotion modulation. To do so, the system of the present invention includes a biophilic sensory unit. The biophilic sensory unit is preferably a large, framed work of preserved plants including, but not limited to, moss and ferns. However, the biophilic units can also be a Vertical Garden (living plant wall). To start the therapeutic experience, the user sits or stands in front of the biophilic sensory unit. Among the plants on the biophilic sensory unit, a matrix barcode is provided that can be scanned by the user using a Personal Computing (PC) device. The matrix barcode can be any type of barcode including, but not limited to, a Quick Response (QR) code that connects to a webpage where several audio recordings are accessible for the user to listen to. In the preferred embodiment, each audio recording is a script that guides the user through various emotional regulation techniques that use of sound effects, tones, music, and spoken words to visually explore the three-dimensional elements of the biophilic sensory unit while suggesting a path of thinking that will elicit calm and joy. The scripts provide an educational component which teaches the different behavior and cognitive modification skills. The scripts are unique to the present invention and are created under the supervision of a psychiatrist to allow the user to move away from their current state of mind and enter a new state of positive emotions. The user is provided with a new skill.

### BRIEF DESCRIPTION OF THE DRAWINGS

FIG. 1 is a diagram view of the system of the present invention.

FIG. 2 is a flowchart of the overall method of the present invention.

FIG. 3 is a flowchart of the continuation of the overall method of the present invention.

FIG. 4 is a flowchart of the subprocess of enabling the user to choose the audio recording to be played using the present invention.

FIG. 5 is a flowchart of the subprocess of uploading new audio recordings using the present invention.

FIG. 6 is a flowchart of the subprocess of guiding the user with the emotion regulation instructions using the present invention.

FIG. 7 is a flowchart of the subprocess of playing the sound tones and music using the present invention.

FIG. 8 is a flowchart of the subprocess of playing the sound effects using the present invention.

FIG. 9 is a top-front-perspective schematic view of the biophilic sensory unit of the present invention.

FIG. 10 is a side-rear-perspective schematic view of the biophilic sensory unit of the present invention.

FIG. 11 is a side-front-perspective schematic view of the biophilic sensory unit of the present invention.

FIG. 12 is a magnified schematic view of the biophilic sensory unit of the present invention, wherein a user scanning the matrix barcode is shown.

FIG. 13 is a magnified schematic view of the biophilic sensory unit of the present invention, wherein the matrix barcode is shown.

### DETAIL DESCRIPTIONS OF THE INVENTION

All illustrations of the drawings are for the purpose of describing selected versions of the present invention and are not intended to limit the scope of the present invention.

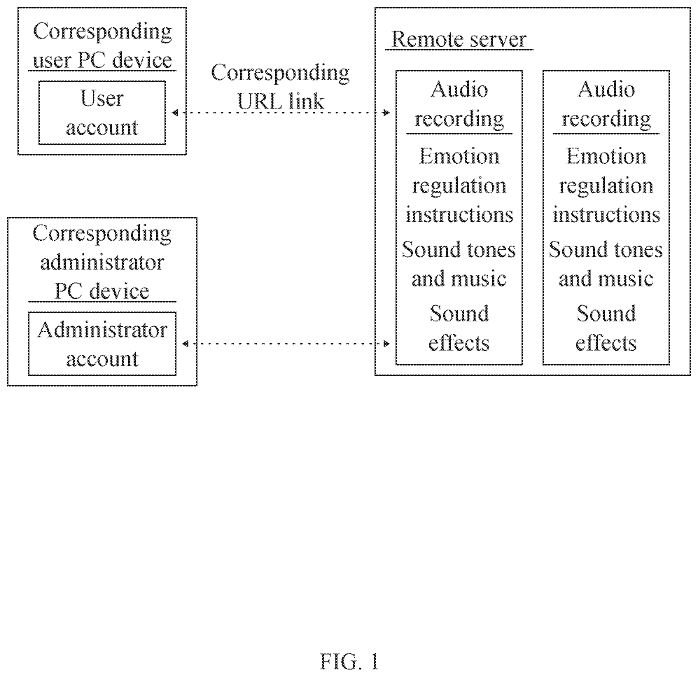

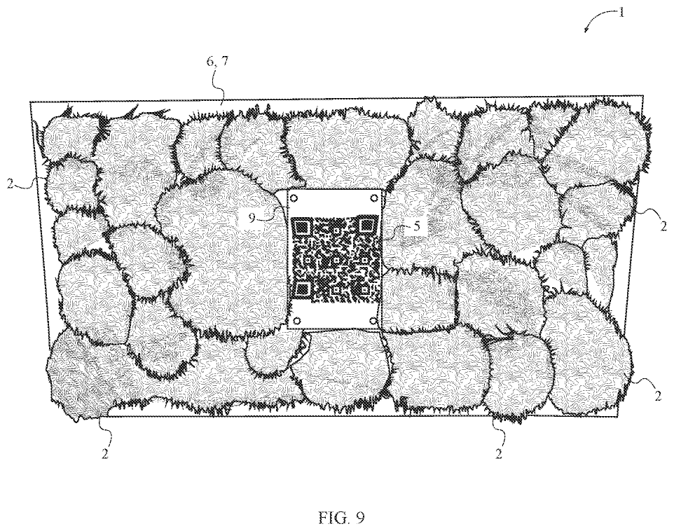

The present invention is a system and a method for facilitating accessible therapeutic intervention to regulate emotions, establish well-being, and provide lifelong behavioral and cognitive skills by synergizing biophilic stimuli with cognitive engagement. The present invention provides the user with immediate stress reduction, calm, as well as lifelong skills for ongoing emotional health and well-being using biophilic sensory attributes. As can be seen in FIGS. 1 and 9, the system of the present invention includes a biophilic sensory unit 1 (Step A). The biophilic sensory unit 1 is preferably a large, framed work of preserved plants including, but not limited to, moss and ferns. However, the biophilic sensory unit may also be a living plant wall. Further, the biophilic sensory unit 1 includes at least one matrix barcode 5 which is associated with a corresponding uniform resource locator (URL) link. Further, the system of the present invention includes a plurality of audio recordings managed by a remote server (Step B). The plurality of audio recordings can be accessed via the URL link that is obtained from the matrix barcode 5. Furthermore, the system of the present invention includes a user account managed by an external server (Step C). The user account enables the user to interact with the URL link. In addition, the user account is associated with a corresponding user personal computing (PC) device that enables the user to interact with the matrix barcode 5. The corresponding user PC device can be any portable computing device able to scan the matrix barcode 5 including, but not limited to, a smartphone, tablet computer, laptop, etc.

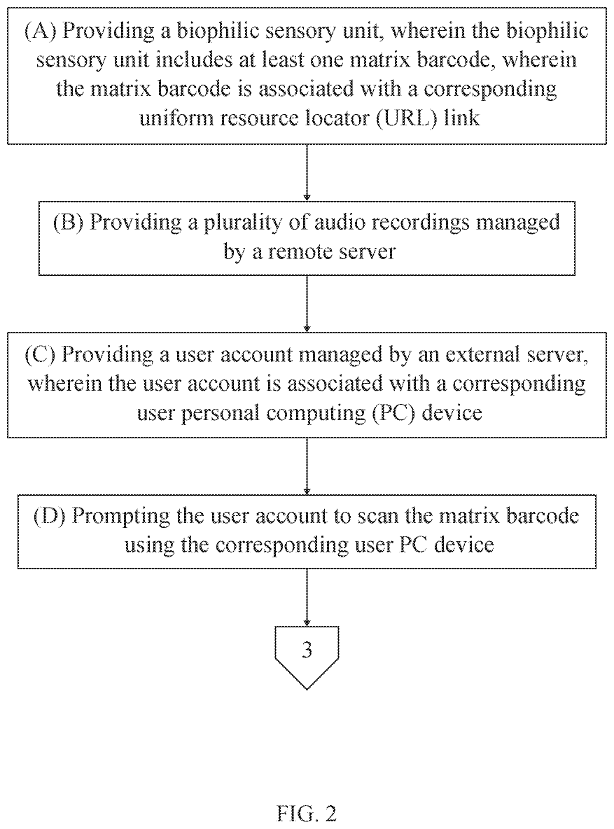

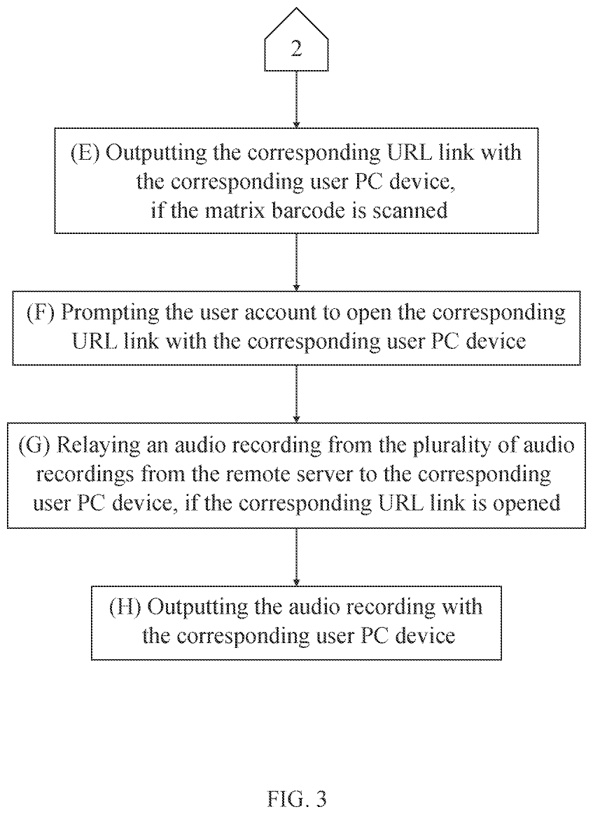

The system of the present invention enables a method that combines tools of psychology and biophilic experiences to help the user achieve immediate stress reduction, calm, as well as lifelong skills for ongoing emotional health and well-being. As can be seen in FIGS. 2 and 3, the overall method of the present invention includes the steps of prompting the user account to scan the matrix barcode 5 using the corresponding user PC device (Step D). For Step D, the user is recommended to be positioned adjacent to the biophilic sensory unit 1, preferably facing the biophilic sensory unit 1, so that the user can receive the biophilic stimuli. Then, the corresponding URL link is output with the corresponding user PC device (Step E), if the matrix barcode 5 is scanned. The user can use the image capturing capabilities of the corresponding user PC device, such as the camera of a smartphone, to scan the matrix barcode 5 on the biophilic sensory unit 1. Then, the user account is prompted to open the corresponding URL link with the corresponding user PC device (Step F). In other words, the user is presented with the URL link on the display of the corresponding user PC device which the user can choose to open. The URL link can be opened using a third-party internet browser to launch the website that is linked to the URL link. Once the corresponding website is launched, the user is presented with the choice to play the desired audio recording from the plurality of audio recordings. So, an audio recording from the plurality of audio recordings is relayed from the remote server to the corresponding user PC device (Step G), if the corresponding URL link is opened. Then, once the chosen audio recording is relayed to the corresponding user PC device, the audio recording is output with the corresponding user PC device (Step H). The audio recording is output with the corresponding user PC device using the device's audio playing capabilities, such as the speakers or headphones of a smartphone, to play the audio recordings. This way, the user can receive sensory input of both sight and sound to allow the user to move away from their current state of mind, enter a new state of positive emotions, and be open to cognitive engagement.

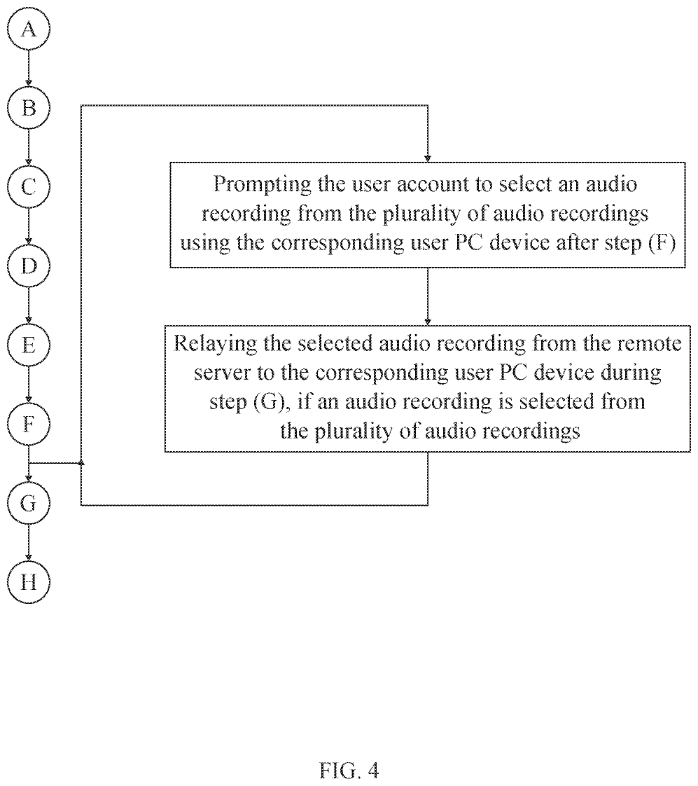

In the preferred embodiment, the plurality of audio recordings includes several pre-recorded scripts that are created, written, and recorded exclusively for the purpose of the present invention. In some embodiments, the user can be given the option to choose the audio recording the user wants to listen to. As can be seen in FIG. 4, the subprocess of enabling the user to choose the audio recording to be played includes the steps of prompting the user account to select an audio recording from the plurality of audio recordings using the corresponding user PC device after Step F. Once the user opens the URL link and the corresponding website is launched, the user is given the option to select an audio recording from the plurality of audio recordings currently available. Then, the selected audio recording is relayed from the remote server to the corresponding user PC device during Step G, if an audio recording is selected from the plurality of audio recordings. In other words, only the selected audio recording is relayed to the corresponding user PC device to be listened to by the user. This way, the user can select the audio recording the user wants to listen to during each biophilic therapy session to enjoy a unique therapeutic experience each time.

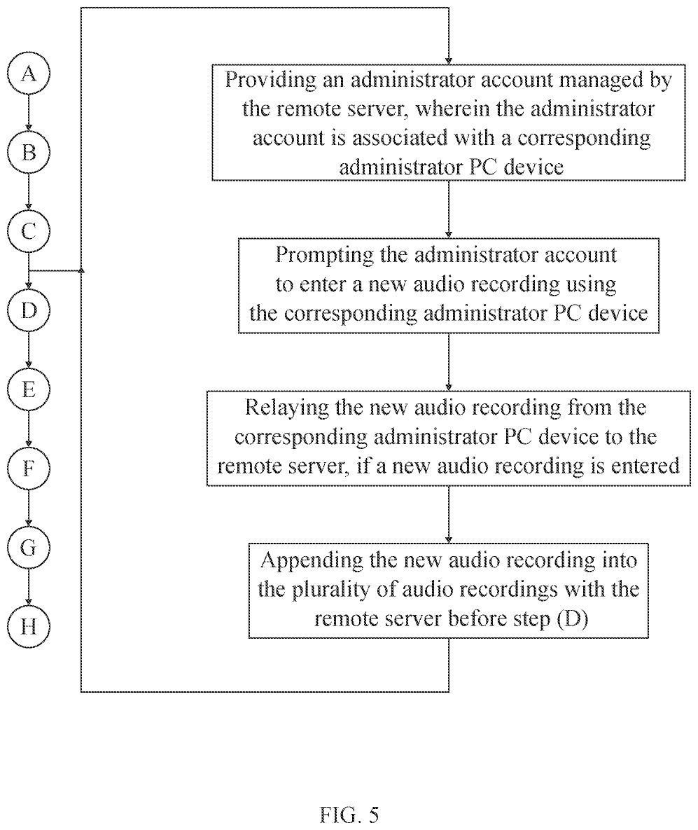

To provide consistent users with new audio recordings on every biophilic therapy session, the system of the present invention enables new audio recordings to be uploaded to the remote server. As can be seen in FIG. 1, the system of the present invention may further include an administrator account managed by the remote server. The administrator account enables administrators of the present invention to manage the audio recordings available to the users. Further, the administrator account is associated with a corresponding administrator PC device that enables the administrators to upload new audio recordings to the remote server. As can be seen in FIG. 5, the subprocess of uploading new audio recordings includes the steps of prompting the administrator account to enter a new audio recording using the corresponding administrator PC device. The new audio recording can be recorded using the corresponding administrator PC device or can be recorded using the third-party system and then transferred to the corresponding administrator PC device. Then, the new audio recording is relayed from the corresponding administrator PC device to the remote server, if a new audio recording is entered. Further, the new audio recording is appended into the plurality of audio recordings with the remote server before Step D. This way, the new audio recording is stored in the remote server to be readily available to the user whenever the user wants to experience the biophilic therapy. In some embodiments, the new audio recordings can replace the old audio recordings so that only current audio recordings are available to the user. In other embodiments, all current and previous audio recordings are stored in the remote server so that the user can choose any audio recording that has been uploaded to the remote server.

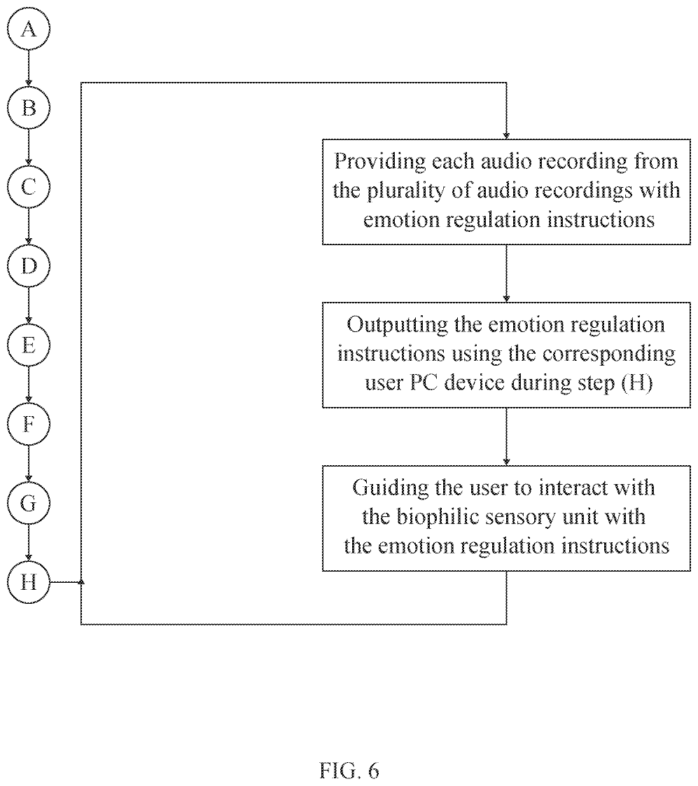

As previously discussed, the plurality of audio recordings includes different therapeutical recordings that complement the biophilic sensory unit 1. So, each audio recording from the plurality of audio recordings is preferably provided with emotion regulation instructions. The emotion regulation instructions guide the user to visually explore the three-dimensional elements of the constructed nature design of the biophilic sensory unit 1 while suggesting a path of thinking that will elicit calm and joy. As can be seen in FIG. 6, the subprocess of guiding the user with the emotion regulation instructions includes the steps of outputting the emotion regulation instructions using the corresponding user PC device during Step H. The emotion regulation instructions change according to the chosen audio recording from the plurality of audio recordings. Then, the user is guided to interact with the biophilic sensory unit 1 with the emotion regulation instructions. Furthermore, the emotion regulation instructions can utilize Cognitive Behavior Therapy (CBT) and/or Dialectical Behavior Therapy (DBT) approaches and techniques to identify and change negative thinking patterns and direct positive behavioral changes. These CBT and DBT techniques are integrated with the recognized positive effects of the human experience with nature (biophilia), where these biophilic experiences reduce stress, improve cognitive function, and enhance mood and creativity. So, the emotion regulation instructions can include cognitive-behavior-therapy-based scripts and/or dialectical-behavior-therapy-based scripts. In alternate embodiments, different approaches and techniques can be utilized to generate and provide the emotion regulation instructions.

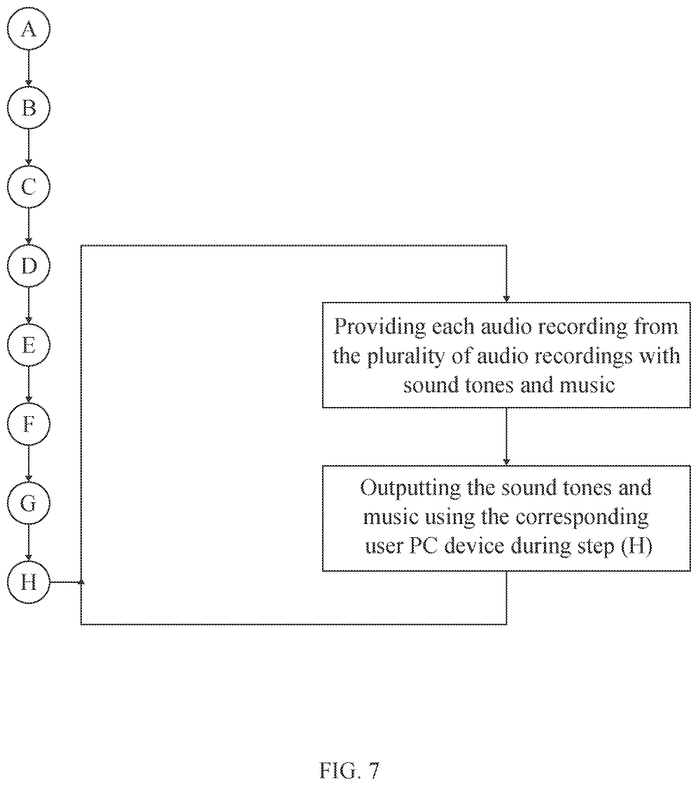

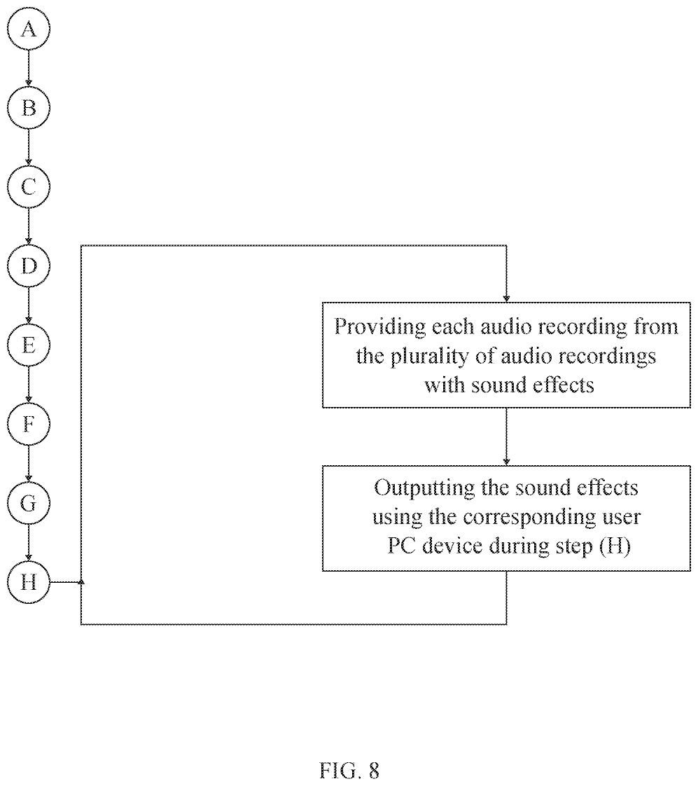

In addition to the emotion regulation instructions, the plurality of audio recordings can include other auditory stimuli to improve the therapeutic experience of the user. In one embodiment, each audio recording from the plurality of audio recordings is provided with sound tones and music. As can be seen in FIG. 7, the subprocess of playing the sound tones and music includes the step of outputting the sound tones and music using the corresponding user PC device during Step H. This way, the user can listen to the emotion regulation instructions along with the sound tones and music while experiencing the therapeutic session. In another embodiment, each audio recording from the plurality of audio recordings can be provided with sound effects. As can be seen in FIG. 8, the subprocess of playing the sound effects includes the step of outputting the sound effects using the corresponding user PC device during Step H. Thus, the user can be provided with different auditory stimuli while following the emotion regulation instructions. For example, an audio recording from the plurality of audio recordings can include the following scripts:

### Example: Rotating Session—Thoughts and Emotions Script

Nature Sounds

### Speaker/Voice

### Metered, Thoughtful Pace

Welcome to the present invention, your access to the essential skills of being. This session, Thoughts and Emotions, introduces the skillful action of telling our thoughts apart from our emotions. Being able to tell the difference between a thought and an emotion allows us to become more attuned with our thinking and our feelings and to understand how they impact each other. Now, let's begin.

Bell Chime
Find a comfortable spot facing the plants on the wall. Take a moment to reflect on how your day is going so far. Think about the interactions you've had with your coworkers/your boss/your family/your friends.

Pause—2 Seconds
Consider one interaction that stands out to you. Consider what sets this interaction apart.

Pause—2 Seconds
Maybe it stands out because of how it made you feel. Emotions are experiences we feel in our body and are labeled based on their context. We can have the same feeling in our body that means different things depending on the context. A racing heart can signal danger/excitement/or joy. Butterflies in our stomach can signal worry/or anticipation. Thinking back to the interaction that stood out to you from earlier, think about how you felt in your body before/during/and after.

Pause—2 Seconds
Think about how you feel now. Notice whether the sensations in your body come back when you imagine this interaction.

Pause—3 Seconds
Emotions, or feelings, often come in response to triggers around us or inside of us. Triggers inside of us may be thoughts—the conversations/statements/or evaluations we make inside our head. Thoughts can be put into words or described to another person. They can bring about sensations in our body.

Pause—2 Seconds
Consider the interaction you reimagined earlier—what do you think about that person? If you think “I like them,” there may be a sensation that arises in your body—perhaps your heart fluttering/or a sense of peace. If you think “I do not like them,” the sensation that arises may be similar, but conveys a sense of irritation or stress. If you think “they were wearing blue shoes today,” you might not have any sensation in your body. This is a neutral thought—a way to describe this person that is not emotionally charged.

Pause—2 Seconds
Thoughts can impact our feelings. And our emotions can impact our thoughts. Our feelings are important signals, but sometimes our thoughts become inaccurate because of the strength of our feelings.

Pause—2 Seconds.
The next time you are feeling a surge of feeling, of emotion in your body, consider where you feel the sensations in your body. Consider what happened before that surge of feeling and if you can describe the thoughts you had before and after.

Pause—2 Seconds.
Congratulations. You have completed the session, Thoughts and Emotions. You may practice this skill whenever you′d like, noticing the sensations/in your body/and the thoughts/in your mind.

### Example: Always Accessible Session—Grounding Your Roots

Nature Sounds

### Speaker/Voice

### Metered, Thoughtful Pace

Welcome to the present invention, your access to the essential skills of being. This brief session, Grounding Your Roots, can be used when you are feeling emotionally or mentally overwhelmed. It is designed to quickly orient you to your body/your space/and to give a sense of calm/and control. Let's begin.

Bell Chime
Position yourself in front of the plant wall so that you can clearly see the details of one section. We will begin by taking two/deep breaths/together. We will inhale for three/two/one, and exhale slowly for three/two/one. Good. Now, let us inhale once more for three/two/one, and exhale slowly for three/two/one.

Pause—2 Seconds
Set aside any thoughts that you have swirling in your mind. And let your eyes rest on an area of the plant wall/taking in the details of the plants/and the moss.

Pause—2 Seconds
To yourself, name three things that you see on the wall. You may say them quietly to yourself or mouth the words soundlessly.

Pause—5 Seconds
Let us take a deep breath together again. Inhale for three/two/one. And exhale slowly for three/two/one. Notice any thoughts/in your mind. Notice your body/and any sensations you feel.

Pause—5 Seconds
With your attention directed towards your body/make note of three things that you can feel/or touch. Perhaps you can feel your clothes/the ground/beneath/your feet/the air on your face.

Pause—5 Seconds
And just/continue/to breathe.

Pause—10 Seconds
This concludes your grounding exercise. If you are not yet feeling more at ease, you may repeat this exercise again/continuing/to breathe and setting aside your thoughts for just/a brief/moment.

Bell Chime—End

In other embodiments, the plurality of audio recordings can include other auditory stimuli to enhance the therapeutic experience of the user.

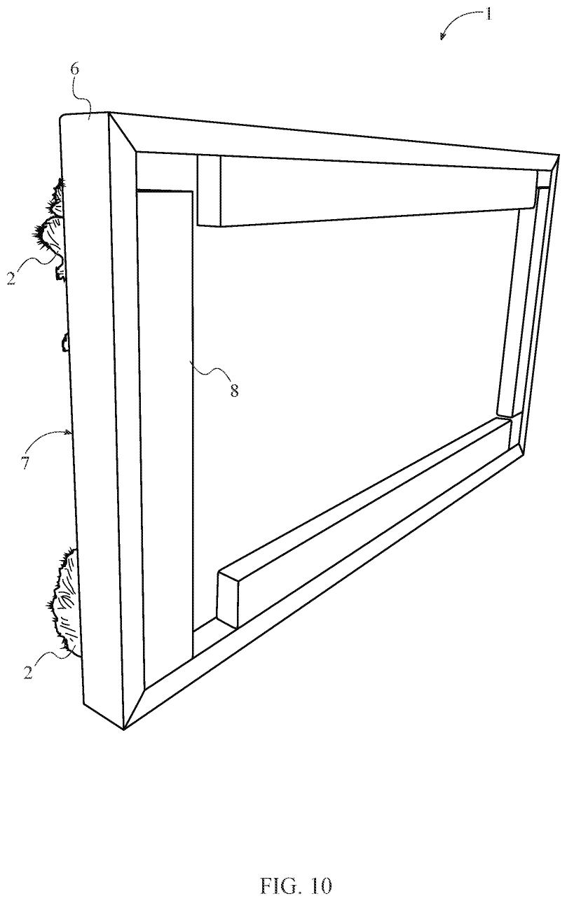

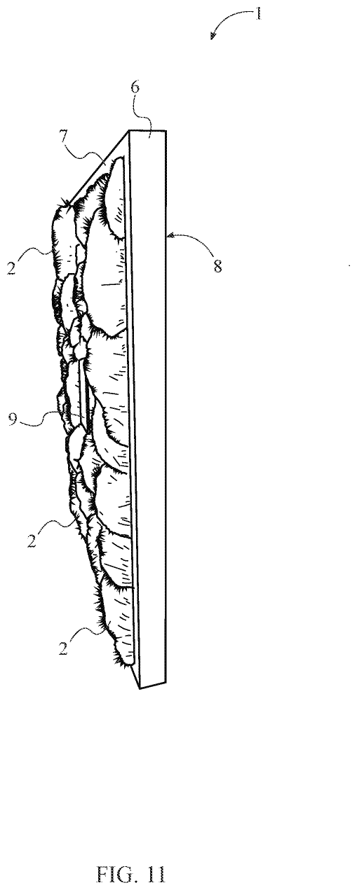

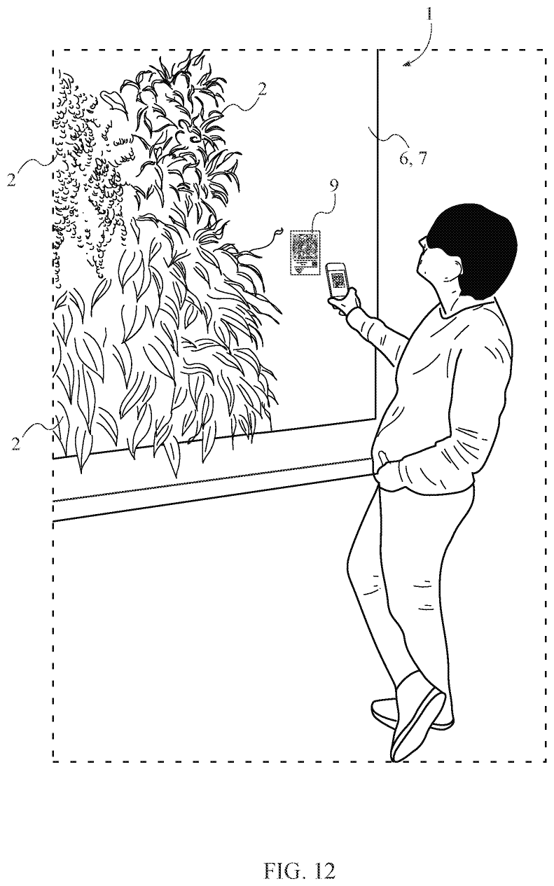

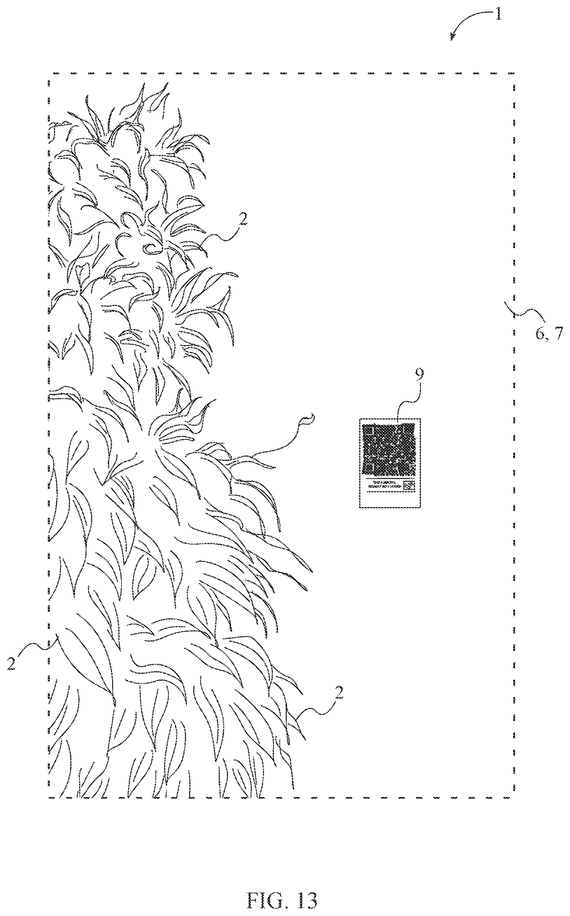

As previously discussed, the system of the present invention includes a biophilic sensory unit 1 that the user can visually inspect and experience during the therapeutic session. In the preferred embodiment, the biophilic sensory unit 1 comprises a plurality of preserved plants 2, a matrix barcode 5, and a support board 6, as can be seen in FIG. 9 through 11. The support board 6 is preferably a rectangular structure made from corrugated plastic and pine frame to form a light and sturdy support structure for the plurality of preserved plants 2. Accordingly, the support board 6 comprises a first board face 7 and a second board face 8 corresponding to the opposite flat surfaces of the support board 6. Further, the biophilic sensory unit 1 is assembled as follows. The matrix barcode 5 is positioned among the plurality of preserved plants 2 so that the matrix barcode 5 is not blocked by the plurality of preserved plants 2. Further, the plurality of preserved plants 2 and the matrix barcode 5 are mounted onto the first face to secure the plurality of preserved plants 2 and the matrix barcode 5 to the support board 6. Furthermore, the support board 6 can include a hanging mechanism that enables the support board 6 to be hung from a surface such as a wall. The hanging mechanism can include, but is not limited to, hooks that are secured to the second board face 8 that can be engaged with fasteners on the desired surface. Thus, the biophilic sensory unit 1 can be securely hung from the desired surface to be accessible to any user. In other embodiments, the matrix barcode 5 can be positioned on the periphery of the plurality of preserved plants 2, as can be seen in FIGS. 12 and 13.

In the preferred embodiment, the plurality of preserved plants 2 can include several preserved plants that are appropriate for biophilic therapy. As can be seen in FIG. 9 through 13, the plurality of preserved plants 2 can include, but is not limited to, preserved mosses and ferns of different types (e.g., pole, Provence, lycopodium, sheet mosses, Argyria, asparagus, brake, brilliant, common ferns, etc.). Accordingly, the plurality of preserved plants 2 may comprise a plurality of preserved mosses 3 and a plurality of preserved ferns 4. To increase the biophilic stimuli of the biophilic sensory unit 1, the plurality of preserved mosses 3 and the plurality of preserved ferns 4 are distributed throughout the first board face 7 so that plurality of preserved plants 2 covers the whole first board face 7 surface. In addition, the plurality of preserver mosses is interspersed among the plurality of preserved ferns 4 so that the plurality of preserved plants 2 provides the best biophilic stimuli. In other embodiments, the plurality of preserved plants 2 can include different preserved plants or living plants. In an alternate embodiment, the biophilic sensory unit 1 can be provided as a vertical garden (living plant wall) or garden.

In some embodiments, the matrix barcode 5 can be protected to prevent damage to the matrix barcode 5 from the environment or accidents. As can be seen in FIG. 9 through 13, the system of the present invention may further include a barcode casing 9. The barcode casing 9 can be a transparent acrylic frame that protects the matrix barcode 5 while enabling the user to scan the matrix barcode 5. Accordingly, the matrix barcode 5 is positioned within the barcode casing 9 so that the matrix barcode 5 is protected by the barcode casing 9. Further, the matrix barcode 5 is mounted to the first board face 7 by the barcode casing 9 to secure the barcode casing 9 and the matrix barcode 5 to the support board 6. In other embodiments, the matrix barcode 5 can be protected using different mechanisms.

Although the invention has been explained in relation to its preferred embodiment, it is to be understood that many other possible modifications and variations can be made without departing from the spirit and scope of the invention as hereinafter claimed.

What is claimed is:

1. A method for facilitating accessible therapeutic intervention to regulate emotions and establish immediate and long-term well-being by synergizing biophilic stimuli with cognitive engagement to amplify benefits, the method comprising the steps of:
(A) providing a biophilic sensory unit, wherein the biophilic sensory unit includes at least one integrated matrix barcode, wherein the matrix barcode is associated with a corresponding uniform resource locator (URL) link;
(B) providing a plurality of audio recordings managed by a remote server;
(C) providing a user account managed by an external server, wherein the user account is associated with a corresponding user personal computing (PC) device;
(D) prompting the user account to scan the matrix barcode using the corresponding user PC device;
(E) outputting the corresponding URL link with the corresponding user PC device, if the matrix barcode is scanned;
(F) prompting the user account to open the corresponding URL link with the corresponding user PC device;
(G) relaying an audio recording from the plurality of audio recordings from the remote server to the corresponding user PC device, if the corresponding URL link is opened; and
(H) outputting the audio recording with the corresponding user PC device.

2. The method as claimed in claim 1 comprising the steps of:
prompting the user account to select an audio recording from the plurality of audio recordings using the corresponding user PC device after step (F); and
relaying the selected audio recording from the remote server to the corresponding user PC device during step (G), if an audio recording is selected from the plurality of audio recordings.

3. The method as claimed in claim 1 comprising the steps of:
providing an administrator account managed by the remote server, wherein the administrator account is associated with a corresponding administrator PC device;
prompting the administrator account to enter a new audio recording using the corresponding administrator PC device;
relaying the new audio recording from the corresponding administrator PC device to the remote server, if a new audio recording is entered; and
appending the new audio recording into the plurality of audio recordings with the remote server before step (D).

4. The method as claimed in claim 1 comprising the steps of:
providing each audio recording from the plurality of audio recordings with emotion regulation instructions;
outputting the emotion regulation instructions using the corresponding user PC device during step (H); and
guiding the user to interact with the biophilic sensory unit with the emotion regulation instructions.

5. The method as claimed in claim 4, wherein the emotion regulation instructions are cognitive-behavior-therapy-based scripts.

6. The method as claimed in claim 4, wherein the emotion regulation instructions are dialectical-behavior-therapy-based scripts.

7. The method as claimed in claim 1 comprising the steps of:
providing each audio recording from the plurality of audio recordings with sound tones and music; and
outputting the sound tones and music using the corresponding user PC device during step (H).

8. The method as claimed in claim 1 comprising the steps of:
providing each audio recording from the plurality of audio recordings with sound effects; and
outputting the sound effects using the corresponding user PC device during step (H).

9. A system for facilitating accessible therapeutic intervention to regulate emotions and establish immediate and long-term well-being by synergizing biophilic stimuli with cognitive engagement to amplify benefits, the system comprising:
a biophilic sensory unit;
the biophilic sensory unit comprising a plurality of preserved plants, a matrix barcode, and a support board;
the support board comprising a first board face and a second board face;
the matrix barcode being positioned among the plurality of preserved plants; and
the plurality of preserved plants and the matrix barcode being mounted onto the first face.

10. The system as claimed in claim 9 comprising:
the plurality of preserved plants comprising a plurality of preserved mosses and a plurality of preserved ferns;
the plurality of preserved mosses and the plurality of preserved ferns being distributed throughout the first board face; and
the plurality of preserver mosses being interspersed among the plurality of preserved ferns.

11. The system as claimed in claim 9 comprising:
a barcode casing;
the matrix barcode being positioned within the barcode casing; and
the matrix barcode being mounted to the first board face by the barcode casing.
